(19) United States
(12) Patent Application Publication
(10) Pub. No.: US 2022/0072553 A1
(43) Pub. Date: Mar. 10, 2022

(54) DEVICE AND METHOD FOR DETECTING TUMOR MUTATION BURDEN (TMB) BASED ON CAPTURE SEQUENCING

(71) Applicant: ZHENYUE BIOTECHNOLOGY JIANGSU CO., LTD., Taizhou (CN)

(72) Inventors: Hexin SHI, Taizhou (CN); Jianing YU, Taizhou (CN); Yuanyuan HONG, Taizhou (CN); Minjun CHEN, Taizhou (CN); Ying YANG, Taizhou (CN); Junyan HOU, Taizhou (CN); Hong LV, Taizhou (CN); Weizhi CHEN, Taizhou (CN); Shan ZHENG, Taizhou (CN); Ji HE, Taizhou (CN); Bo DU, Taizhou (CN)

(73) Assignee: ZHENYUE BIOTECHNOLOGY JIANGSU CO., LTD., Taizhou (CN)

(21) Appl. No.: 17/202,372

(22) Filed: Mar. 16, 2021

Related U.S. Application Data
(63) Continuation of application No. PCT/CN2021/074742, filed on Feb. 2, 2021.

(30) Foreign Application Priority Data
Sep. 7, 2020 (CN) 202010927039.3

(51) Int. Cl. B01L 3/00; G16B 30/00; C12Q 1/6869; G16B 20/20

(52) U.S. Cl. CPC B01L 3/502784; G16B 20/20; C12Q 1/6869; G16B 30/00

(57) ABSTRACT

A device and a method for detecting tumor mutation burden (TMB) based on capture sequencing are disclosed. The device includes: a panel design module configured to uniformly add population single-nucleotide polymorphism (SNP) sites to a genome and screen out gene regions that show the highest consistency with whole exome sequencing (WES); a data acquisition module configured to acquire tissue and plasma samples of a target object and acquire sequencing data of the samples; an alignment module configured to align the sequencing data with a reference genome to acquire mutation data results; a somatic mutation analysis module configured to perform somatic analysis on the mutation data results to obtain somatic mutation results; a filtering module configured to remove unreal mutation sites from the somatic mutation results; and a calculation module configured to calculate the TMB.

### CROSS REFERENCE TO THE RELATED APPLICATIONS

This application is a continuation application of the International Application PCT/CN2021/074742, filed on Feb. 2, 2021, which is based upon and claims priority to Chinese Patent Application No. 202010927039.3, filed on Sep. 7, 2020, the entire contents of which are incorporated herein by reference.

### TECHNICAL FIELD

The present invention relates to the technical field of biomedicine, and in particular to a device and a method for detecting tumor mutation burden (TMB).

### BACKGROUND

Tumor mutation burden (TMB) or tumor mutation load (TML) is a quantifiable biomarker that reflects the number of mutations in tumor cells, which is usually measured as the number of mutations per million bases in coding regions of a tumor cell genome.

At present, the detection of TMB mainly relies on next-generation sequencing (NGS), and in the gold standard, whole exome sequencing (WES) is used to count and calculate the number of mutations in CDS region (protein coding regions, exons) sequences ≥30 Mb. However, WES has technical problems such as high price, low detection depth, and possible missed detection of low-coverage loci. Therefore, researchers are actively exploring methods based on capture sequencing (panel) to detect TMB, thereby effectively reducing the sequencing cost. However, when TMB is detected using a panel-based method great challenges are faced in accuracy and reliability. At present, there are still deficiencies, such as insufficient consistency between panel and WES, inaccurate detection results when there is no control samples, detection of tumor tissue or plasma TMB of a tumor patient alone, poor specificity to samples with different sequencing depths, and poor specificity to samples with different tumor fractions.

### SUMMARY

To solve the above problems, the present invention provides a device and a method for detecting TMB based on capture sequencing, which effectively solves problems in existing detection technology, for example, there is a low consistency between panel and WES, a tumor tissue or plasma TMB can only be detected alone for a tumor patient, and so on.

The present invention provides the following technical solutions.

The present invention provides a device for detecting TMB based on capture sequencing, including:

a panel design module configured to uniformly add population single-nucleotide polymorphism (SNP) sites to a genome and screen out gene regions that show the highest consistency with WES;

a data acquisition module configured to acquire tissue and plasma samples of a target object and acquire sequencing data of the tissue and plasma samples based on the gene regions screened out by the panel design module;

an alignment module configured to align the sequencing data acquired by the data acquisition module with a reference genome to acquire mutation data results;

a somatic mutation analysis module configured to perform somatic analysis on the mutation data results obtained by the alignment module to obtain somatic mutation results;

a filtering module configured to remove unreal mutation sites from the somatic mutation results obtained by the somatic mutation analysis module to obtain real mutation sites; and

a calculation module configured to calculate the TMB according to the number of real somatic mutation sites obtained by the filtering module.

The present invention also provides a method for detecting TMB based on capture sequencing, including:

uniformly adding population SNP sites to a genome and screening out gene regions that show the highest consistency with WES;

acquiring tissue and plasma samples of a target object and acquiring sequencing data of the tissue and plasma samples based on the gene regions screened out;

aligning the sequencing data with a reference genome to acquire mutation data results;

performing somatic analysis on the mutation data results to obtain somatic mutation results;

removing unreal mutation sites from the somatic mutation results to obtain real mutation sites; and

calculating the TMB according to the number of real somatic mutation sites.

The present invention also provides a terminal device including a memory, a processor, and computer programs that are stored in the memory and can be running on the processor, and when the computer programs are running on the processor, the steps of the method for detecting TMB based on capture sequencing described above are implemented.

The present invention also provides a computer-readable storage medium storing computer programs, and when the computer programs are executed by a processor, the steps of the method for detecting TMB based on capture sequencing described above are implemented.

The device and method for detecting TMB based on capture sequencing provided by the present invention can improve the specificity, accuracy, and reliability of a panel design on the premise of fully improving the consistency between designed panel and WES in the detection of TMB, especially the detection accuracy in the case where there is no control sample results; and can simultaneously detect the TMBs of tumor tissue and plasma in a tumor patient. Specifically, in panel design, germline mutations can be subtracted more accurately by uniformly adding enough population SNP sites. A screening method based on new regions of machine learning is used to select a gene region set with the highest consistency with WES. In addition, a specific baseline is built for different sequencing depths, different sample types, and different tumor fraction intervals to improve the adaptability and accuracy of detection. Moreover, sequence-specific errors (SSEs), sequencing or experimental background noise, black-listed mutations, panel of normal (PoN) sites, etc. are subtracted to obtain highly-reliable somatic mutation information. Furthermore, sequencing data of tissue samples and plasma samples can be obtained at the same time, which realizes the simultaneous TMB detection for tissue and plasma samples of a target object, with high accuracy.

### BRIEF DESCRIPTION OF THE DRAWINGS

Preferred implementations will be described below in a clear and easy-to-understand manner in conjunction with the accompanying drawings to further illustrate the above-mentioned characteristics, technical features, advantages, and implementation methods.

FIG. 1 is a schematic structural diagram of the device for detecting TMB based on capture sequencing according to the present invention;

FIG. 2 is a schematic flow chart of the method for detecting TMB based on capture sequencing according to the present invention;

FIG. 3 is a flow chart of TMB detection in an example of the present invention;

FIG. 4 is a schematic diagram illustrating the consistency between TMB results obtained from WES and panel sequencing in an example of the present invention; and

FIG. 5 is a schematic structural diagram of the terminal device according to the present invention.

### REFERENCE NUMERALS

100 represents a device for detecting TMB, 110 represents a panel design module, 120 represents a data acquisition module, 130 represents an alignment module, 140 represents a somatic mutation analysis module, 150 represents a filtering module, and 160 represents a calculation module.

### DETAILED DESCRIPTION OF THE EMBODIMENTS

In order to explain the examples of the present invention or the technical solutions in the prior art more clearly, the specific implementations of the present invention will be described below with reference to the accompanying drawings. Apparently, the accompanying drawings in the following description show merely some examples of the present invention, and other drawings and other implementations may be derived from these drawings by a person of ordinary skill in the art without creative efforts.

As shown in FIG. 1, a first example of the present invention provides a device 100 for detecting TMB based on capture sequencing, including: a panel design module 110 configured to uniformly add population SNP sites to a genome and screen out gene regions that show the highest consistency with WES; a data acquisition module 120 configured to acquire tissue and plasma samples of a target object and acquire sequencing data of the tissue and plasma samples based on the gene regions screened out by the panel design module 110; an alignment module 130 configured to align the sequencing data acquired by the data acquisition module 120 with a reference genome to acquire mutation data results; a somatic mutation analysis module 140 configured to perform somatic analysis on the mutation data results obtained by the alignment module 130 to obtain somatic mutation results; a filtering module 150 configured to remove unreal mutation sites from the somatic mutation results obtained by the somatic mutation analysis module 140 to obtain real mutation sites; and a calculation module 160 configured to calculate the TMB according to the number of real somatic mutation sites obtained by the filtering module 150.

In this example, the panel design module 110 is configured to screen out gene regions that show the highest consistency with WES to form a panel and includes a uniform site design unit and a region screening unit. The uniform site design unit is configured to screen out genome regions for designing probes according to a first preset rule and then uniformly add population SNP sites screened out according to a second preset rule to accurately subtract germline mutations. The region screening unit is configured to screen out the gene regions that show the highest consistency with WES by machine learning on exons.

In actual situations, blood cell data of a patient are often not available and TMB only considers somatic mutations, so most TMB methods involve no germline control data. Therefore, in this example, in order to improve the accuracy of a process where an in silico algorithm is used to remove possible germline mutations, enough population SNP sites are uniformly added in the panel design stage. Specifically, the design includes the following steps:

1.1 Genome regions for designing probes are screened as follows: removing gaps and regions with a mappability lower than 40 in a genome; after a genome is divided according to a preset window (such as 200 bp and 300 bp) and step size (such as 1 bp and 2 bp), removing regions that have a GC content higher than 30% and lower than 60%;

1.2 removing regions that include a preset number (such as 3) of sites with an Asian population heterozygosity greater than a preset threshold (such as 0.5 and 0.6) or more and have a corresponding preset length (such as 120 bp).

1.3 SNP sites in a 1,000 genomes database in the regions for designing probes are screened as follows:

I) SNP sites with an Asian population heterozygosity greater than a specified threshold (such as 0.5 and 0.6);

II) SNP sites that meet Hardy-Weinberg equilibrium;

III) extending an SNP site on both sides to a sufficient size (for example, a fixed size of 100 bp, trying to make the SNP site in the middle of a region) to facilitate the design of probes; and

IV) using existing mature tools (such as BWA and BLAST) to align the extended region with a human reference genome sequence, counting the number of positions in each region that can be aligned to the genome, and removing regions in which the number is greater than a preset threshold (such as 10).

Furthermore, the step of filtering based on heterozygosity and Hardy-Weinberg equilibrium is as follows:

1) downloading SNP data of a 1,000 genomes phase 3;

2) using existing mature tools (such as plink) to calculate the minor allele frequency (MAF) in the EAS population (Asian population data in the 1,000 genomes database) for each population polymorphic site and the pvalue of Hardy-Weinberg equilibrium;

3) screening out sites where the pvalue of Hardy-Weinberg equilibrium is greater than a specified fixed threshold (such as 0.05 and 0.06); and

4) screening out population polymorphic sites with high MAF in the EAS population.

In order to design a panel with the highest consistency with WES, a screening process of the region screening unit includes:

2.1 For any cancer, DNA mutation data corresponding to the cancer are downloaded in TCGA or other public databases (or self-produced sample databases).

2.2 A human genome reference sequence (hg19) and corresponding annotation files are downloaded; according to location information of the annotation files, the number of mutations on each exon of each sample is counted (excluding pathogenic mutations such as cosmic); and an exon length is standardized.

2.3 A TMB value on WES (denoted as TMB wes) is calculated for each sample.

2.4 Exons whose GC content and mappability cannot meet the requirements for designing probes are removed (for example, regions with a GC content higher than 30% and lower than 60% are removed).

2.5 The machine learning method is used to rank all exons and mark them as exon (1), exon (2), exon (3), . . . , exon (N), where, N is the number of exons included in the analysis.

TMB-high (such as TMB>10/Mb) and TMB-low (such as TMB<5/Mb) tumor samples are selected for exon ranking. A ranking method specifically includes: randomly selecting a specified percentage of samples (such as 70% and 80%) each time for feature screening, and repeating multiple times (such as 100 times and 150 times); counting the number of times that each exon is selected; and ranking the exons based on the counted number of times from largest to smallest. Feature selection can be achieved by methods such as random forest, logistics regression, and backward stepwise regression, and AIC test criteria. In the case where the random forest method is used, if the exons each are selected the same number of times, the exons can also be ranked based on importance from largest to smallest.

2.6 After the exons are ranked based on importance, starting from the most important exon (1), a marked exon is added in sequence, a TMB value of each exon set is calculated, and a consistency of the TMB value with a TMB result obtained from WES is evaluated (when TCGA data are downloaded, a consistency with a TMB result of TCGA WES is evaluated); and when a specified consistency threshold is reached, or the consistency cannot be effectively improved by adding exon, or a set region size is almost the maximum acceptable region size, the calculation is stopped and the region is regarded as a gene region with the highest consistency with WES. Specific steps are as follows:

I) a selected exon region set is denoted as exon set, and in the i-th round, exon_set={exon(1), . . . , exon(i)};

II) a calculated sample includes only a TMB value of the exon set (denoted as TMB_select_i);

III) If one of the following conditions is met, the cycle is stopped:
a) a correlation cor(i) between TMB_select_i and TMB_wes is greater than a specified threshold (such as R\hat{ }2>0.9);

b) a difference between cor(i) and cor(i−1) is less than a specified threshold (such as 0.0001); and
c) a total length of exons included in exon_set is greater than a specified threshold (such as 10 M);

IV) if the cycle is not stopped in step III), exon_set={exon(1), . . . , exon(i), exon(i+1)} is set, and the steps I) to IV) are repeated until the cycle is stopped in step III).

It should be noted that an optional determination method for b) in step III) includes: directly calculating a correlation for all exon combinations under the ranking and displaying results as a curve graph; and when the correlation meets the convergence condition visually at a given number of exons, an exon combination meeting the convergence condition is selected as a gene region with the highest consistency with WES.

The data acquisition module 120 may include an acquisition unit and a quality control unit. The acquisition unit may be configured to acquire raw data of tissue and plasma samples of a target object, and the quality control unit may be configured to perform quality control on the raw data of tissue and plasma samples separately to obtain sequencing data. The alignment module 130 may include a first alignment unit and a second alignment unit. The first alignment unit may be configured to align the sequencing data with a reference genome to obtain an alignment result file, and the second alignment unit may be configured to subject the alignment result file to remove redundancy and to re-alignment in terms of InDel regions to obtain mutation data results. In one example, the first alignment unit is configured to use the bwa software to align sequencing data that meet the data sequencing quality and sequencing data quality with the human reference genome hg19, and then use the samtools software to sort bam to obtain mutation data results; and the second alignment unit is configured to use the GATK and picard tools to perform de-redundancy and re-alignment of InDel regions.

In another example, the device 100 for detecting TMB further includes a specific baseline building module configured to build different sequencing depth baselines and tumor fraction baselines for different sequencing depth intervals, sample types, and tumor fraction intervals. Considering that there may be different biases in coverage and different BAF-0.5 deviations at the germline SNP site for different sequencing depths or sample types, in this example, different baselines are built for different sequencing depths or sample types to be used, thus achieving superior adaptability and accuracy. In addition, given the difference in detection frequency caused by different tumor fractions in different tissue sample pathological sections, in this example, different frequency baselines are built for different tumor fraction intervals, thereby facilitating more sensitive and accurate identification of real mutations in tissue samples with different purities. In one example, different tumor fractions of existing tumor samples are divided into multiple gradients in the pathological evaluation: 0% to 10%, 10% to 20%, 20% to 30%, and 30% or more, and then baselines are built for different tumor fraction intervals, so that the TMB algorithm is suitable for pathological samples with different tumor fractions.

Based on this, in the somatic mutation analysis module 140, when there are samples for control analysis, VarDict or MuTect2 is used to subject the mutation data results acquired by the alignment module 130 to somatic analysis to obtain somatic mutation results. When there is no sample for control analysis, a corresponding sequencing depth baseline is selected according to a sequencing depth and a sample type of tissue and plasma samples, and somatic mutation results are acquired based on an in silico germline subtraction algorithm.

Specifically, steps of the in silico germline subtraction algorithm include:

3.1 Third-party software such as MuTect2 is used to detect all candidate micromutations, including somatic single-base variants (SNVs) and germline single-base variants (SNPs).

3.2 Rolling median, locally weighted regression (LWR), and other methods are used to acquire coverage, and GC correction is conducted.

3.3 Healthy people/known negative FFPE samples are used to build a baseline distribution (baseline1) of coverage under different sequencing depths and sample types.

3.4 Healthy people/known negative FFPE samples are used to build BAF baselines of heterozygous SNPs under different sequencing depths and sample types, and specifically, software such as GATK is used to detect a genotype of each sample at each SNP site, and a distribution baseline2_1 of heterozygous SNP BAF (mean value μ, standard deviation σ, excluding heterozygous SNPs with μ that significantly deviates from 0.5 or has too-large variance), a distribution baseline2_2 of homozygous SNP BAF, and a distribution baseline2_3 of non-mutant BAF are counted, separately.

3.5 The baseline1 corresponding to depth/sample type is used to calculate a log-ratio of a copy number of each capture region in a to-be-tested sample.

3.6 The circular binary segmentation (CBS) method is used to conduct segmentation on the log-ratio for each region above. For ease of expression, it is assumed that L segmented regions (segments) are obtained. In an example, it can be weighted CBS, for example, a reciprocal of a standard deviation of a coverage in a healthy population is adopted as a weight.

3.7 Each of the obtained segments is further subjected to fine segmentation based on SNP sites thereon:

a) SNP sites must meet the following filtering conditions: max{baseline2_3}+k*σ<BAF<min{baseline2_2}−k*σ for a to-be-tested sample, k=0, 1, 2, or 3, and a coverage depth is greater than a specified threshold (such as 100);

b) each BAF is converted into z-mBAF according to formula (1);

z−mBAF=abs(BAF−μ)/σ (1)
c) based on z-mBAF, the CBS method is used to obtain new segments, and it is assumed that M segments are finally obtained.

3.8 On the basis of PureCN, ASCAT, and other methods, the grid search method is used to estimate multiple sets of local optimal solutions for tumor purity (β) and ploidy (Ψ), and a posterior probability is calculated for the copy number and BAF under different combinations.

Definition: mBAF=min{abs(BAF−μ)+μ,100}. Log-ratio (rᵢ) and mBAF (bᵢ) are used for evaluation, where, i represents the i-th segment, and the expectations of the variables rᵢ and bᵢ are shown in formulas (2) and (3):

$$\begin{matrix}E [ {r}_{i} ] = {log}_{2} ( \frac{2 * ( 1 - ρ ) + ρ * {C}_{i}}{ρ * Ψ + 2 * ( 1 - ρ )} ) & ( 2 ) \\ E [ {b}_{i} ] = \frac{1 - ρ + ρ * {n}_{B , i}}{2 - 2 * ρ + ρ * C} & ( 3 )\end{matrix}$$

where, Cᵢ represents the copy number, Cᵢ=n_{A,i}+n_{B,i}; and n_{A,i} and n_{B,i} represent the copy numbers of two alleles, respectively.

3.9 For all the segments, the least squares method is used to obtain solutions for ρ and Ψ, the copy number-based information (formula 2) and SNP-based information (formula 3) are estimated, and different weights are given.

3.10 According to the multiple local optimal purity and ploidy combinations estimated and the segment divisions, software such as PureCN is used to determine a somatic status of each candidate SNV. Basic principles: log-likelihood is first calculated for each candidate SNV according to beta distribution, a score is calculated thereby for each purity and ploidy combination, and then the combinations are ranked. Usually, a purity and ploidy combination with the highest score is finally selected, or a combination ranking the second/third is selected based on experience.

After somatic mutation results are obtained by analysis of the somatic mutation analysis module 140, unreal mutation sites are filtered out by the filtering module 150 according to annotation results of the somatic mutation results obtained by the somatic mutation analysis module 140 to obtain real mutation sites with a quantity of Mn. Specifically, a filtering rule may include: removing in silico germline mutations according to sample types; filtering out sites with mutation frequency less than 5% and prevalance more than 0.2% in a population database; filtering out known tumor-driven gene mutations; filtering out mutation sites manifested as non-germline sites with high population frequency; filtering out repeat regions or false positive sites generated from alignment of homologous regions according to a pre-built noise baseline of FFPE sample feature SSE; filtering out PoN sites with a frequency less than a sum of a mean value and 5-fold standard deviation for PoN sites; filtering out preset black-listed sites, namely, sites that have an occurrence frequency greater than 30% in populations or have a population frequency greater than 20% in two sample types of FFPE samples, plasma samples, and blood cell samples; and/or screening out mutations that meet depth requirements according to a sequencing depth baseline and screening out mutations that meet a tumor fraction according to a tumor fraction baseline. In one example, Mutect2 is used to perform somatic analysis on the mutation data results, and after vcf file results (somatic mutation results) are obtained, the annovar software is used to annotate to obtain database annotation results; and then annotated sites are filtered by the filtering module 150.

Specifically, in this process, in order to strictly control mutation sites included in the calculation, false positive sites are filtered out by simultaneously considering mutations caused by sequencing or experimental background noise and SSEs, PoN, and site blacklists to finally obtain highly-reliable somatic mutation information. The process to strictly control mutation sites included in the calculation mainly includes the following steps:

4.1 Background Noise

According to the frequency (greater than or equal to 0.1%) distribution of mutation sites among a specified number (such as 30) of normal people, a one-sided 95% confidence interval is selected as a threshold of background noise, and sample sites with a mutation frequency greater than or equal to a sum of a mean value and 3-fold standard deviation (mean+3sd) are retained.

4.2 Filtering Out of False Positive Mutations Caused by SSEs

Mutation sites manifested as non-germline sites with high population frequency, repeat regions, or false positive sites generated from alignment of homologous regions are filtered out. SSE is strictly filtered out by building a noise baseline of FFPE sample feature SSE.

4.3 PoN

The same experiment and analysis process are adopted to count an occurrence frequency of mutation sites among a specified number (such as 30) of normal human blood cell and plasma samples. A site occurring in two or more normal people is regarded as a PoN site. For mutations within a PoN range, if an actual detection frequency is greater than or equal to a sum of a mean value and 5-fold standard deviation for PoN sites, it is retained, otherwise, it is filtered out.

4.4 Blacklist

A specified number (such as 1,000) of FFPE samples, plasma samples, and blood cell samples are taken from an internal database to build a mutation blacklist. Sites with an occurrence frequency greater than 30% in populations or a population frequency greater than 20% in any two sample types are selected as black-listed sites, which will be filtered out directly.

The calculation module 160 is configured to calculate the TMB according to the number of real somatic mutation sites obtained by the filtering module 150, as shown in formula (4):

TMB=Mn/Tn*1000000 (4)

where, Tn represents the number of mutation sites in all mutation data.

In the above examples, deficiencies in the current TMB detection methods are overcomed, such as poor specificity, low consistency, low reliability, inaccurate detection results when there is no control samples, and detection of tumor tissue or plasma TMB of a tumor patient alone. Under the premise of fully improving the consistency between designed panel and WES in the detection of TMB, the present invention comprehensively improves the accuracy of each link, especially improves the specificity, accuracy, and reliability of the panel design. The present invention also can increase the detection accuracy when there is no control sample. Moreover, the present invention improves the detection accuracy for special tissue or plasma samples with different depths, different purities, and different tumor fractions, and provides a more targeted, sensitive, and accurate detection device for the calculation of TMB.

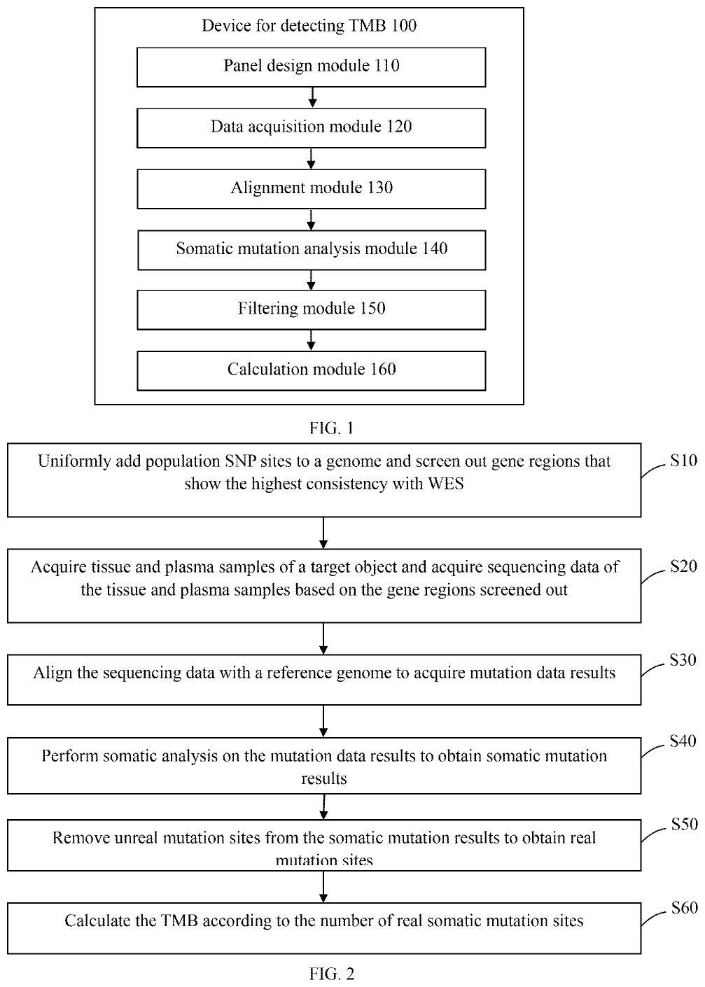

As shown in FIG. 2, another example of the present invention provides a method for detecting TMB based on capture sequencing, which can be used on the device for detecting TMB described above. The method for detecting TMB includes: S10 uniformly adding population SNP sites to a genome and screening out gene regions that show the highest consistency with WES; S20 acquiring tissue and plasma samples of a target object and acquiring sequencing data of the tissue and plasma samples based on the gene regions screened out; S30 aligning the sequencing data with a reference genome to acquire mutation data results; S40 performing somatic analysis on the mutation data results to obtain somatic mutation results; S50 removing unreal mutation sites from the somatic mutation results to obtain real mutation sites; and S60 calculating the TMB according to the number of real somatic mutation sites.

In this example, due to actual situations, blood cell data of a patient is often not available and TMB only considers somatic mutations, so most TMB methods involve no germline control data. Therefore, in this example, in order to improve the accuracy of a process where an in silico algorithm is used to remove possible germline mutations, enough population SNP sites are uniformly added in the panel design stage. Specifically, the design includes the following steps:

1.1 Genome regions for designing probes are screened as follows: removing gaps and regions with a mappability lower than 40 in a genome; after a genome is divided according to a preset window (such as 200 bp and 300 bp) and step size (such as 1 bp and 2 bp), removing regions that have a GC content higher than 30% and lower than 60%;

1.2 removing regions that include a preset number (such as 3) of sites with an Asian population heterozygosity greater than a preset threshold (such as 0.5 and 0.6) or more and have a corresponding preset length (such as 120 bp).

1.3 SNP sites in a 1,000 genomes database in the regions for designing probes are screened as follows:

I) SNP sites with an Asian population heterozygosity greater than a specified threshold (such as 0.5 and 0.6);

II) SNP sites that meet Hardy-Weinberg equilibrium;

III) extending an SNP site on both sides to a sufficient size (for example, a fixed size of 100 bp, trying to make the SNP site in the middle of a region) to facilitate the design of probes; and

IV) using existing mature tools (such as BWA and BLAST) to align the extended region with a human reference genome sequence, counting the number of positions in each region that can be aligned to the genome, and removing regions in which the number is greater than a preset threshold (such as 10).

In order to design a panel with the highest consistency with WES, a screening process of the region screening unit includes:

2.1 For any cancer, DNA mutation data corresponding to the cancer are downloaded in TCGA or other public databases (or self-produced sample databases).

2.2 A human genome reference sequence (hgl9) and corresponding annotation files are downloaded; according to location information of the annotation files, the number of mutations on each exon of each sample is counted (excluding pathogenic mutations such as cosmic); and exon lengths are normalized.

2.3 A TMB value on WES (denoted as TMB_wes) is calculated for each sample.

2.4 Exons whose GC content and mappability cannot meet the requirements for designing probes are removed (for example, regions with a GC content higher than 30% and lower than 60% are removed).

2.5 The machine learning method is used to rank all exons and mark them as exon (1), exon (2), exon (3), . . . , exon (N), where, N is the number of exons included in the analysis.

TMB-high (such as TMB>10/Mb) and TMB-low (such as TMB<5/Mb) tumor samples are selected for exon ranking. A ranking method specifically includes: randomly selecting a specified percentage of samples (such as 70°/s and 80%) each time for feature screening, and repeating multiple times (such as 100 times and 150 times); counting the number of times that each exon is selected; and ranking the exons based on the counted number of times from largest to smallest. Feature selection can be achieved by methods such as random forest, logistics regression, and backward stepwise regression, and AIC test criteria. In the case where the random forest method is used, if the exons each are selected the same number of times, the exons can also be ranked based on importance from largest to smallest.

2.6 After the exons are ranked based on importance, starting from the most important exon (1), a marked exon is added in sequence, a TMB value of each exon set is calculated, and a consistency of the TMB value with a TMB result obtained from WES is evaluated (when TCGA data are downloaded, a consistency with a TMB result of TCGA WES is evaluated); and when a specified consistency threshold is reached, or the consistency cannot be effectively improved by adding exon, or a set region size is almost the maximum acceptable region size, the calculation is stopped and the region is regarded as a gene region with the highest consistency with WES. Specific steps are as follows:

I) a selected exon region set is denoted as exon set, and in the i-th round, exon_set={exon(1), . . . , exon(i)};

II) a calculated sample includes only a TMB value of the exon set (denoted as TMB_select_i);

III) If one of the following conditions is met, the cycle is stopped:

a) a correlation cor(i) between TMB_select_i and TMB_wes is greater than a specified threshold (such as R\hat{ }2>0.9);

b) a difference between cor(i) and cor(i−1) is less than a specified threshold (such as 0.0001); and

c) a total length of exons included in exon set is greater than a specified threshold (such as 10 M);

IV) if the cycle is not stopped in step III), exon_set={exon(1), . . . , exon(i),exon(i+1)} is set, and the steps I) to IV) are repeated until the cycle is stopped in step III).

It should be noted that an optional determination method for b) in step Ill) includes: directly calculating a correlation for all exon combinations under the ranking and displaying results as a curve graph; and when the correlation meets the convergence condition visually at a given number of exons, an exon combination meeting the convergence condition is selected as a gene region with the highest consistency with WES.

In step S20, quality control is performed on the obtained raw data of tissue and plasma samples of the target object separately to obtain sequencing data. In step S30, the sequencing data are first aligned with a reference genome to obtain an alignment result file, and then the alignment result file is subjected to de-redundancy and re-alignment in terms of InDel regions to obtain mutation data results. In one example, the bwa software is used to align sequencing data that meet the data sequencing quality and sequencing data quality with the human reference genome hg19, and then the samtools software is used to sort bam to obtain mutation data results; and the GATK and picard tools are used to perform de-redundancy and re-alignment of InDel regions.

In another example, the method for detecting TMB based on capture sequencing further includes the step of building different sequencing depth baselines and tumor fraction baselines for different sequencing depth intervals, sample types, and tumor fraction intervals. Specifically, considering that there may be different biases in coverage and different BAF-0.5 deviations at the germline SNP site for different sequencing depths or sample types, in this example, different baselines are built for different sequencing depths or sample types to be used, thus achieving superior adaptability and accuracy. In addition, given the difference in detection frequency caused by different tumor fractions in different tissue sample pathological sections, in this example, different frequency baselines are built for different tumor fraction intervals, thereby facilitating more sensitive and accurate identification of real mutations in tissue samples with different purities. In one example, different tumor fractions of existing tumor samples are divided into multiple gradients in the pathological evaluation: 0% to 10%, 10% to 20%, 20% to 30%, and 30% or more, and then baselines are built for different tumor fraction intervals, so that the TMB algorithm is suitable for pathological samples with different tumor fractions.

Based on this, in step S40, when there are samples for control analysis, VarDict or MuTect2 is used to subject the mutation data results to somatic analysis to obtain somatic mutation results. When there is no sample for control analysis, a corresponding sequencing depth baseline is selected according to a sequencing depth and a sample type of tissue and plasma samples, and somatic mutation results are acquired based on an in silico germline subtraction algorithm.

Specifically, steps of the in silico germline subtraction algorithm include:

3.1 Third-party software such as MuTect2 is used to detect all candidate micromutations, including somatic single-base variants (SNVs) and germline single-base variants (SNPs).

3.2 Rolling median, LWR, and other methods are used to acquire coverage, and GC correction is conducted.

3.3 Healthy people/known negative FFPE samples are used to build a baseline distribution (baseline1) of coverage under different sequencing depths and sample types.

3.4 Healthy people/known negative FFPE samples are used to build BAF baselines of heterozygous SNPs under different sequencing depths and sample types, and specifically, software such as GATK is used to detect a genotype of each sample at each SNP site, and a distribution baseline2_1 of heterozygous SNP BAF (mean value μ, standard deviation σ, excluding heterozygous SNPs with μ that significantly deviates from 0.5 or has too-large variance), a distribution baseline2_2 of homozygous SNP BAF, and a distribution baseline2_3 of non-mutant BAF are counted, separately.

3.5 The baseline1 corresponding to depth/sample type is used to calculate a log-ratio of a copy number of each capture region in a to-be-tested sample.

3.6 The CBS method is used to conduct segmentation on the log-ratio for each region above. For ease of expression, it is assumed that L segments are obtained. In an example, it can be weighted CBS, for example, a reciprocal of a standard deviation of a coverage in a healthy population is adopted as a weight.

3.7 Each of the obtained segments is further subjected to fine segmentation based on SNP sites thereon:

a) SNP sites must meet the following filtering conditions: max {baseline2_3}+k*σ<BAF<min{baseline2_2}−k*σ for a to-be-tested sample, k=0, 1, 2, or 3, and a coverage depth is greater than a specified threshold (such as 100);

b) each BAF is converted into z-mBAF according to formula (1);

c) based on z-mBAF, the CBS method is used to obtain new segments, and it is assumed that M segments are finally obtained.

3.8 On the basis of PureCN, ASCAT, and other methods, the grid search method is used to estimate multiple sets of local optimal solutions for tumor purity (P) and ploidy), and a posterior probability is calculated for the copy number and BAF under different combinations.

Definition: mBAF=min{abs(BAF−μ)+μ,100}. Log-ratio (rᵢ) and mBAF (bᵢ) are used for evaluation, where, i represents the i-th segment, and the expectations of the variables rᵢ and bᵢ are shown in formulas (2) and (3).

3.9 For all the segments, the least squares method is used to obtain solutions for ρ and Ψ, the copy number-based information (formula 2) and SNP-based information (formula 3) are estimated, and different weights are given.

3.10 According to the multiple local optimal purity and ploidy combinations estimated and the segment divisions, software such as PureCN is used to determine a somatic status of each candidate SNV. Basic principles: log-likelihood is first calculated for each candidate SNV according to beta distribution, a score is calculated thereby for each purity and ploidy combination, and then the combinations are ranked. Usually, a purity and ploidy combination with the highest score is finally selected, or a combination ranking the second/third is selected based on experience.

In step S50, after somatic mutation results are obtained, unreal mutation sites are filtered out according to annotation results of the somatic mutation results to obtain real mutation sites with a quantity of Mn. Specifically, a filtering rule may include: removing in silico germline mutations according to sample types; filtering out sites with mutation frequency less than 5% and prevelance more than 0.2% in a population database; filtering out known tumor-driven gene mutations; filtering out mutation sites manifested as non-germline sites with high population frequency; filtering out repeat regions or false positive sites generated from alignment of homologous regions according to a pre-built noise baseline of FFPE sample feature SSE; filtering out PoN sites with a frequency less than a sum of a mean value and 5-fold standard deviation for PoN sites; filtering out preset black-listed sites, namely, sites that have an occurrence frequency greater than 30% in populations or have a population frequency greater than 20% in two sample types of FFPE samples, plasma samples, and blood cell samples; and/or screening out mutations that meet depth requirements according to a sequencing depth baseline and screening out mutations that meet a tumor fraction according to a tumor fraction baseline. In one example, Mutect2 is used to perform somatic analysis on the mutation data results, and after vcf file results (somatic mutation results) are obtained, the annovar software is used to annotate to obtain database annotation results; and then in step S50, annotated sites are filtered. In step S60, TMB is calculated according to the number of real somatic mutation sites obtained by the filtering module, as shown in formula (4).

In one example:

1. Sequencing Library Construction

Based on NGS, library construction was conducted for tissue samples (FFPE), plasma samples, and blood cell samples (BC) according to the following steps (the blood cell samples required no interruption treatment):

1. Sample Interruption:

Polytetrafluoroethylene (PTFE) threads were cut into a length of about 1 cm using ultraviolet (UV)-sterilized medical scissors, ensuring that the interruption rods had uniform lengths, and then the PTFE threads were placed in a clean container and UV-sterilized for 3 h to 4 h. After the sterilization was completed, the 1 cm PTFE threads were transferred to a 96-well plate with sterilized tweezers, with 2 interruption rods for each well, and then the 96-well plate was UV-sterilized for 3 h to 4 h.

300 ng of an FFPE/bc DNA sample was taken according to qubit quantitative results, diluted to 50 μl with TE, and transferred to a 96-well plate; a tin foil membrane was placed on the 96-well plate, with four sides of the membrane being aligned with that of the plate; heat sealing was conducted at 180° C. for 5 s twice with a heat sealer; and then the plate was centrifuged in a microplate centrifuge.

A preset program Peak Power was selected: 450; Duty Factor: 30; Cycles/Burst: 200; Treatment time: 40 s, 3 cycles; and then “Start position” was clicked. A “Run” button on a Run interface was clicked to run the program. After the program was completed, the sample plate was take out, centrifuged with a microplate centrifuge, and then placed on a sample holder, and a program Peak Power was selected: 450; Duty Factor: 30; Cycles/Burst: 200; Treatment time: 40 s, 4 cycles. A “Run” button on a Run interface was clicked to run the program. After the program was completed, the sample plate was taken out and centrifuged with a microplate centrifuge. After interruption, 1 μl was taken for quality control.

2. Library Preparation Steps:

An end was repaired and an A tail was added at the 3′ end: ER & AT Mix was prepared according to Table 1 below.

TABLE 1
ER & AT Mix preparation

| Reagent | Volume | |
|---|---|---|
| End Repair & A-Tailing Buffer | 7 | μL |
| End Repair & A-Tailing Enzyme Mix | 3 | μL |
| Total volume | 10 | μL |

10 μL pf ER & AT Mix was taken and added to a DNA sample (operating on ice), and a resulting mixture was shaken for thorough mixing and centrifuged for a short time. Note: immediately after ER & AT Mix and DNA were thoroughly mixed by vortexing, PCR was conducted. A reaction system was placed on a PCR instrument, and PCR was conducted according to the following table. Here, a heated lid temperature of the PCR instrument was set to 85° C. If the experimental procedure shown in Table 2 below is conducted immediately after the above operation is completed, a termination temperature should be set to 20° C.

TABLE 2
Experimental conditions for end repair and A-tailing

| Step | Temperature | Time |
|---|---|---|
| End Repair | 20° C. | 30 min |
| and A-Tailing | 65° C. | 30 min |
| Termination | 20° C. | ∞ |

Linking Adapter:

Adapter preparation: 2.5 μL of IDT UDI adapter was diluted to 5 μL with 2.5 μL of water. Ligation Mix preparation (operating on ice): According to the number of libraries, Ligation Mix was prepared according to Table 3 below and shaken for thorough mixing.

TABLE 3
Ligation Mix preparation

| Reagent | Volume | |
|---|---|---|
| Ultrapure water (UPW) | 5 | μL |
| Ligation Buffer | 30 | μL |
| DNA Ligase | 10 | μL |
| Total volume | 45 | μL |

After the PCR in the previous step was completed, the sample was taken out, centrifuged for a short time, and transferred to a diluted adapter solution. Then 45 μL of Ligation Mix was added, and a resulting mixture was shaken for thorough mixing, then centrifuged for a short time, placed on a PCR instrument, incubated at 20° C. for 30 min, and stored at 20° C. A heated lid temperature was set to 50° C. Purification after ligation: After the PCR in the previous step was completed, the sample was taken out and centrifuged for a short time, and 88 μL of magnetic beads was added. A resulting mixture was shaken for thorough mixing (a tube cap was tightened when shaking), and incubated at room temperature for 15 min so that DNA was fully bound to the magnetic beads. A resulting mixture was centrifuged for a short time and placed on a magnetic separator until a clear supernatant was obtained, and a supernatant was discarded (no magnetic beads were pipetted). 200 μL of 80% ethanol was added for 30 s of incubation and then removed. The washing was repeated once with 200 μL of 80% ethanol (prepared just before use). A 10 μL pipette tip was used to completely remove residual ethanol at the bottom of the centrifuge tube. The magnetic beads were dried at room temperature for 3 min to 5 min until the ethanol was completely volatilized (a front side was not reflective and a back side was dry). Note: excessive drying of magnetic beads will result in reduction of a DNA yield. The centrifuge tube was taken off from the magnetic separator, 22 μL of UPW was added, and a resulting mixture was shaken for thorough mixing (a tube cap was tightened when shaking), incubated at room temperature for 5 min, centrifuged for a short time, and placed on a magnetic separator until a clear supernatant was obtained. 1 μL of DNA library was taken for concentration detection, and the remaining 20 μL of supernatant was transferred to a new PCR tube for the next amplification test.

Library amplification: PCR Mix was prepared according to Table 4 below (operating on ice), shaken for thorough mixing, centrifuged for a short time, dispensed into 0.2 mL PCR tubes, and stored in a refrigerator at 4° C.

TABLE 4
PC'R Mix preparation

| Reagent | Volume | |
|---|---|---|
| HiFi HotStart ReadyMix (2×) | 25 | μL |
| Library Amplification Primer Mix (10×) | 5 | μL |
| Total volume | 30 | μL |

The library obtained in the previous step was transferred to the dispensed PCR Mix, and a resulting mixture was shaken for thorough mixing, centrifuged for a short time, and placed on a PCR instrument. PCR was conducted according to Table 5 below.

TABLE 5
PCR conditions

| Step | Temperature | | Time | | Number of cycles |
|---|---|---|---|---|---|
| Pre-denaturation | 98° | C. | 45 | s | 1 |
| Denaturation | 98° | C. | 15 | s | 6 to 12 cycles are |
| Annealing | 60° | C. | 30 | s | determined according |
| Extension | 72° | C. | 30 | s | to concentration |
| Re-extension | 72° | C. | 1 | min | 1 |

| | | | | |
|---|---|---|---|---|
| Storage | 8° | C. | ∞ | 1 |

DNA acquisition (1× Beads recovery): After PCR, the sample was taken out and centrifuged for a short time, and 50 μL of Beckman Agencourt AMPure XP magnetic beads was added. A resulting mixture was shaken for thorough mixing (a tube cap was tightened when shaking), and incubated at room temperature for 15 min so that DNA fully bound to the magnetic beads. A resulting mixture was centrifuged for a short time and placed on a magnetic separator until a clear supernatant was obtained, and a supernatant was discarded (no magnetic beads were pipetted). 200 μL of 80% ethanol (prepared just before use) was added for 30 s of incubation and then removed. The washing was repeated once with 200 μL of 80% ethanol. A 10 μL pipette tip was used to completely remove residual ethanol at the bottom of the centrifuge tube. The magnetic beads were dried at room temperature for 3 min to 5 min until the ethanol was completely volatilized (a front side was not reflective and a back side was dry). Note: Excessive drying of magnetic beads will result in reduction of a DNA yield. The centrifuge tube was taken off from the magnetic separator, 40 μL of UPW was added, and a resulting mixture was shaken for thorough mixing and then incubated at room temperature for 5 min to elute DNA. A resulting mixture was centrifuged for a short time and placed on a magnetic separator until a clear supernatant was obtained, and then the library was transferred to a new centrifuge tube and stored at −20° C.

3. Library Quality Control:

1 μL of DNA library was taken for concentration detection. Based on NGS, capturing was conducted for FFPE, plasma, and be samples as follows: 370 genes were selected for WES, with an exon region coverage of 1,684,573 bp. A specific gene list was shown in Table 10.

4. Mixed Library:

Libraries were mixed in equal parts and added to a 1.5 mL centrifuge tube, with a total amount of 1 μg. An added volume of each library was calculated based on the concentration of each library and the number of capture libraries. An added volume of a library: (1,000 ng/number of capture libraries/library concentration) μL. 2.5 μL of Universal Blocking Oligos was added to the above system, then 5 μL of COT Human DNA was added, and a resulting mixture was shaken for thorough mixing and then centrifuged for a short time. The EP tube was sealed with parafilm and then placed in a vacuum centrifugal concentrator for evaporation drying (60° C., about 20 min to 1 h). Note: whether evaporation drying was completed was observed at any time. DNA denaturation: after the evaporation drying was completed for the sample, 7.5 μL of 2× hybridization buffer (vial 5) and 3 μL of hybridization component A (vial 6) were added to each capture, and a resulting mixture was shaken for thorough mixing, centrifuged for a short time, and placed in a heating module at 95° C. for 10 min of denaturation.

5. Hybridization of Library with Probe

The probe was taken out, centrifuged for a short time, and placed in a PCR instrument at 47° C.; the denatured DNA was quickly transferred from 95° C. to the PCR tube with the probe; and a resulting mixture was shaken for thorough mixing, then centrifuged for a short time, and subjected to hybridization at 47° C. in a PCR instrument for no less than 16 h. Preparation of a wash buffer working solution: a buffer required for a capture was prepared by a method shown in Table 6. Buffers were prepared according to Table 6 based on the number of captures.

TABLE 6
Buffer preparation

| | | | 1 × working |
|---|---|---|---|
| Reagent | Reagent/μL | Water/μL | solution volume/μL |
| 10 × Stringent | 40 | 360 | 400 |
| Wash Buffer(vial 4) | | | |
| 10 × Wash | 30 | 270 | 300 |
| Buffed(vial 1) | | | |
| 10 × Wash | 20 | 180 | 200 |
| BufferII( vial 2) | | | |
| 10 × Wash | 20 | 180 | 200 |
| Buffer III (vial 3) | | | |
| 2.5 × Bead | 200 | 300 | 500 |
| Wash Buffer (vial 7) | | | |

Dispensing of reagents to be incubated: 400 μL of 1× stringent wash buffer (vial 4) was dispensed into an 8-tube strip; 100 μL of 1× wash buffer I (vial 1) was dispensed into an 8-tube strip; and 20 μL of capture beads was dispensed into an 8-tube strip. Incubation of capture beads and wash buffer (vial 4 and via 1) working solutions: capture beads must be equilibrated at room temperature for 30 min before use; and Wash buffer (vial 4 and via 1) working solutions must be incubated at 47° C. for 2 h before use.

6. Purification after Hybridization:

100 μL of magnetic capture beads was dispensed for each capture, and 100 μL of magnetic capture beads was placed on a magnetic separator until a clear supernatant was obtained, and the supernatant was discarded. 200 μL of 1× bead wash buffer (vial 7) was added, a resulting mixture was shaken for thorough mixing and placed on a magnetic separator until a clear supernatant was obtained, and the supernatant was discarded. 200 μL of 1× bead wash buffer (vial 7) was added, a resulting mixture was shaken for thorough mixing and placed on a magnetic separator until a clear supernatant was obtained, and the supernatant was discarded. 100 μL of 1× bead wash buffer (vial 7) was added, and a resulting mixture was shaken for thorough mixing and placed on a magnetic separator until a clear supernatant was obtained, and the supernatant was discarded. At this time, the pretreatment of the magnetic beads was completed, and the next test was conducted immediately. A hybridization solution undergoing capture overnight was transferred to the washed magnetic beads, and a resulting mixture was pipetted up and down ten times and incubated at 47° C. for 45 min in a PCR instrument (a PCR heated lid temperature was set to 57° C.), where, the mixture was shaken every 15 min to ensure that the magnetic beads were suspended.

Washing: after the incubation was completed, 100 μL of 1× wash buffer I (vial 1) preheated at 47° C. was added to each tube, and a resulting mixture was shaken for thorough mixing, placed on a magnetic separator until a clear supernatant was obtained, and the supernatant was discarded. 200 μL of 1× stringent wash buffer (vial 4) preheated at 47° C. was added, a resulting mixture was pipetted up and down ten times for thorough mixing, incubated at 47° C. for 5 min, and placed on a magnetic separator until a clear supernatant was obtained, and the supernatant was discarded. Note: a temperature during operation should be kept at 47° C. or above as far as possible. 200 μL of 1× stringent wash buffer (vial 4) preheated at 47° C. was added, a resulting mixture was pipetted up and down ten times for thorough mixing, incubated at 47° C. for 5 min, and placed on a magnetic separator until a clear supernatant was obtained, and the supernatant was discarded. Note: a temperature during operation should be kept at 47° C. or above as far as possible. 200 μL of 1× wash buffer I (vial 1) placed at room temperature was added, a resulting mixture was shaken for 2 min, centrifuged for a short time, and placed on a magnetic separator until a clear supernatant was obtained, and the supernatant was discarded. 200 μL of 1× wash buffer II (vial 2) placed at room temperature was added, a resulting mixture was shaken for 1 min, centrifuged for a short time, and placed on a magnetic separator until a clear supernatant was obtained, and the supernatant was discarded. 200 μL of 1× wash buffer III (vial 3) placed at room temperature was added, a resulting mixture was shaken for 30 s, centrifuged for a short time, and placed on a magnetic separator until a clear supernatant was obtained, and the supernatant was discarded. 20 μL of UPW was added to the centrifuge tube for elution, and a resulting mixture was shaken for thorough mixing and then used for the amplification test in the next step.

7. Post-LM-PCR:

Post-LM-PCR Mix was prepared according to Table 7 and shaken for thorough mixing.

TABLE 7
Post-LM-PCR Mix preparation

| Reagent | Volume | |
|---|---|---|
| HiFi HotStart Ready Mix | 25 | μL |
| Post-LM-PCR Oligos 1 & 2, 5 μM | 5 | μL |
| DNA eluted in the previous step | 20 | μL |
| Total | 50 | μL |

The above sample was transferred to a PCR system, and a resulting mixture was shaken for thorough mixing, centrifuged for a short time, and placed on a PCR instrument. PCR was conducted according to Table 8 below:

TABLE 8
PCR conditions

| Step | Temperature | | Time | | Number of cycles |
|---|---|---|---|---|---|
| Pre-denaturation | 98° | C. | 45 | s | 1 |
| Denaturation | 98° | C. | 15 | s | 13 |
| Annealing | 60° | C. | 30 | s | |
| Extension | 72° | C. | 30 | s | |
| Re-extension | 72° | C. | 1 | min | 1 |

| | | | | |
|---|---|---|---|---|
| Storage | 8° | C. | ∞ | 1 |

Purification after amplification: DNA purification beads were taken out and equilibrated for 30 min at room temperature for later use. 90 μL of purification beads were added to a 1.5 mL centrifuge tube and then 50 μL of amplified capture DNA library was added. A resulting mixture was shaken for thorough mixing, then incubated at room temperature for 15 min, and placed on a magnetic separator until a clear supernatant was obtained, and the supernatant was discarded. 200 μL of 80% ethanol (prepared just before use) was added for 30 s of incubation and then removed. The washing was repeated once with 200 μL of 80% ethanol. A 10 μL pipette tip was used to completely remove residual ethanol at the bottom of the centrifuge tube. The magnetic beads were dried at room temperature until the ethanol was completely volatilized (the magnetic beads were not reflective from a front side and were dry from a back side). Note: excessive drying of magnetic beads will result in reduction of a DNA yield. The centrifuge tube was taken off from the magnetic separator, 50 μL of UPW was added, and a resulting mixture was shaken for thorough mixing and then incubated at room temperature for 2 min. A resulting mixture was centrifuged for a short time and then placed on a magnetic separator until a clear supernatant was obtained, and a capture sample was transferred to a new centrifuge tube.

8. Quality Control:

1 μL of a capture sample was taken for Qubit concentration detection. After the library was qualified, computer sequencing was conducted by nexseq 500 sequencer on the illumina platform, with a sequencing strategy of PE 75 and a data volume of 10 G for each sample.

II. Data Analysis

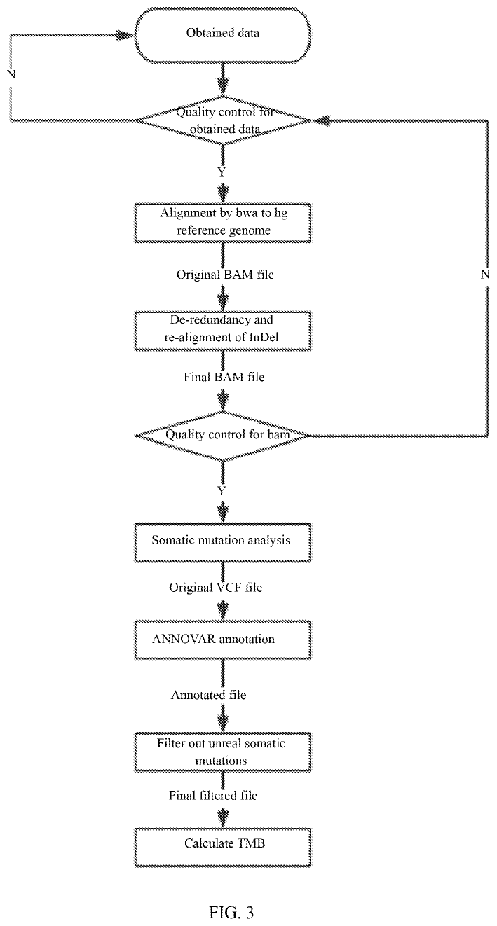

A specific analysis flow chart is shown in FIG. 3:

5.1 It was determined whether the data quality control, data sequencing quality, and total sequencing amount met requirements, if so, clean data was obtained.

5.2 The obtained clean data was aligned to a human reference genome hg19 using bwa, and samtools was used to sort barn files.

5.3 Picard and GATK tools were used to subject obtained barn files to de-redundancy and re-alignment of InDel regions.

5.4 Mutect2/VarDict was used to call mutations using barn files obtained from the re-alignment to somatic mutation analysis to obtain vcf files.

5.5 The obtained vcf files were annotated with the annovar tool to obtain database annotation results.

5.6 From obtained annotated files, sites with mutation frequency less than 5% and prevalance greater than 0.2% in a population database were filtered out; clearly-known tumor-driven gene mutations were filtered out; mutation sites manifested as non-germline sites with high population frequency, repeat regions, or false positive sites generated from alignment of homologous regions were filtered out; SSE was filtered out through a built noise baseline of FFPE sample feature SSE; PoN sites were filtered as follows: mutations in a PoN range that had an actual sample frequency greater than or equal to a sum of a mean value and 5-fold standard deviation for PoN sites were retained; black-listed sites were filtered out; considering a tumor fraction range of samples, in silico germline mutations were subtracted according to different sample types, and mutations meeting depth requirements were screened out according to a sequencing depth baseline.

5.7 The number of somatic mutation sites obtained from the above filtering for final calculation was counted as Mn.

5.8 The samtools tool was applied to the barn files obtained in 5.3 to obtain a coverage depth of each site.

5.9 The total number of mutations in files counted in 5.8 was counted as Tn, and the number of somatic mutation sites obtained from the above filtering for final calculation was counted as Mn.

5.10 TMB was calculated as follows: TMB=Mc/Tn*1000000.

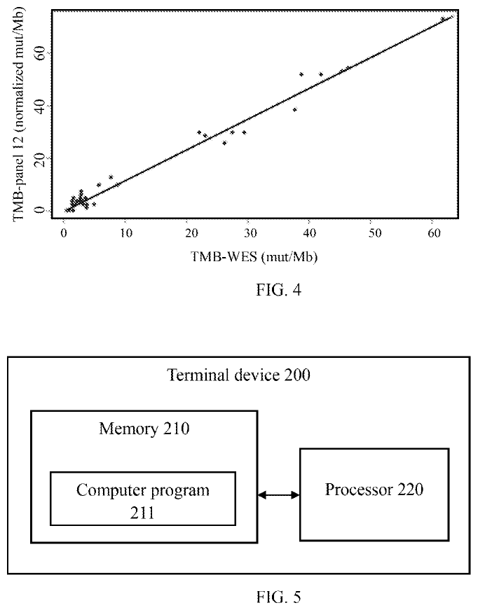

According to the above method, WES and panel capture sequencing were conducted on the tissue samples of 37 patients to analyze the TMB of the patients, and the consistency between TMB results of the 37 patients obtained by WES and panel capture sequencing was analyzed. Results are shown in FIG. 4 (the x-coordinate shows the TMB detected by WES and the y-coordinate shows the TMB detected by panel capture sequencing). It can be seen from the figure that there is a correlation of R\hat{ }2=0.965 between TMB results of the 37 patients obtained from WES and panel capture sequencing. Detailed TMB results are shown in Table 9 below.

TABLE 9
TMB results of 37 patients detected
by WES and panel capture sequencing

| | | TMB detected by |
|---|---|---|
| Sample No. | TMB detected by WES | panel capture sequencing |
| 1 | 0.8884 | 0.01 |
| 2 | 0.7084 | 0.02 |
| 3 | 0.756 | 0.03 |
| 4 | 0.5226 | 0.04 |
| 5 | 1.5833 | 0.05 |
| 6 | 3.7254 | 1.2384 |
| 7 | 3.795 | 2.4756 |
| 8 | 1.4896 | 2.4756 |
| 9 | 3.1881 | 2.4759 |
| 10 | 4.9381 | 2.4761 |
| 11 | 1.4064 | 2.4765 |
| 12 | 2.0177 | 2.4767 |
| 13 | 2.1082 | 3.7141 |
| 14 | 2.5343 | 3.7143 |
| 15 | 1.4658 | 3.7151 |
| 16 | 2.728 | 3.7152 |
| 17 | 3.0367 | 3.7155 |
| 18 | 3.1806 | 3.7184 |
| 19 | 3.5319 | 4.9526 |
| 20 | 1.5729 | 4.9529 |
| 21 | 2.7283 | 4.9534 |
| 22 | 2.8779 | 6.1891 |
| 23 | 2.8117 | 7.4278 |
| 24 | 8.8146 | 9.9032 |
| 25 | 5.7488 | 13.6191 |
| 26 | 7.6891 | 16.1143 |
| 27 | 26.2442 | 23.5287 |
| 28 | 23.0795 | 28.468 |
| 29 | 29.4263 | 29.7153 |
| 30 | 22.0558 | 29.7165 |
| 31 | 27.4723 | 29.7209 |
| 32 | 37.6813 | 30.9515 |
| 33 | 38.7548 | 51.998 |
| 34 | 45.3118 | 53.2259 |
| 35 | 46.3029 | 54.4637 |
| 36 | 41.9442 | 58.2008 |
| 37 | 61.7136 | 73.0266 |

It can be seen from the above results that the method for detecting TMB according to the present invention can not only simultaneously detect tissue and plasma samples, but also lead to detection results with high accuracy.

TABLE 10
List of 370 genes

| | | | | | | | | |
|---|---|---|---|---|---|---|---|---|
| AKT1 | AKT2 | ABL1 | BAP1 | BCL2L1 | PARP3 | HLA-A | FAM175A | ARID1B |
| ALK | AKT3 | AMER1 | BLM | BIVM-ERCC5 | PAX5 | HLA-C | ALOX12B | ARID2 |
| AR | APC | AXIN1 | BMPR1A | BTG1 | PDCD1LG2 | HLA-B | AURKA | ARID5B |
| ARAF | ARID1A | AXIN2 | BRIP1 | CALR | PDPK1 | STK11 | AURKB | ASXL1 |
| BRAF | ATM | CBL | CHEK1 | CARD11 | PIK3C2G | TGFBR2 | AXL | ASXL2 |
| BRCA1 | ATR | CCND2 | EPCAM | CBFB | PIM1 | TSC2 | BCL6 | BCOR |
| BRCA2 | ATRX | CCND3 | ERCC2 | CD79A | PLCG2 | VHL | BRD4 | CASP8 |
| CDK4 | CCND1 | CCNE1 | ERCC3 | CD79B | PPARG | FGF19 | BTK | CDK12 |
| CTNNB1 | CD274 | CDKN1A | ERCC4 | CDC73 | PRDM1 | B2M | CRKL | CEBPA |
| DDR2 | CDH1 | CDKN1B | FANCA | CDK8 | PREX2 | DNMT3A | CTLA4 | CIC |
| EGFR | CDK6 | CDKN2C | FANCC | CRLF2 | PRKAR1A | BARD1 | ERG | CTCF |
| ERBB2 | CDKN2A | CUL3 | FH | CSF3R | PTPRD | FOXO1 | ETV1 | FUBP1 |
| ERBB3 | CDKN2B | E2F3 | GALNT12 | CXCR4 | PTPRS | BCL2 | EZH2 | HNF1A |
| ERRFI1 | CHEK2 | FAT1 | GREM1 | CYLD | PTPRT | NFKBIA | FOXA1 | KDM5C |
| ESR1 | CREBBP | GSK3B | MDC1 | DAXX | RARA | HMCN1 | GLI1 | kDM6A |
| FBXW7 | CSF1R | INPP4B | MRE11A | DICER1 | RFWD2 | KMT2C | IGF1 | KMT2A |
| FGFR1 | EP300 | KDM5A | MUTYH | DIS3 | SDHA | MAP3K1 | IGF2 | KMT2D |
| FGFR2 | ERBB4 | LATS1 | NBN | DNMT1 | SDHAF2 | DDR1 | IRS2 | MAP2K4 |
| FGFR3 | FGF3 | LATS2 | NTHL1 | DNMT3B | SH2D1A | MITF | JUN | NPM1 |
| FLT1 | FGF4 | MAX | PARP1 | DOT1L | SLX4 | MLH3 | MED12 | RBM10 |
| GNA11 | FGFR4 | MDM4 | PMS1 | EED | SOCS1 | MPL | MEF2B | RUNX1 |
| GNAQ | FLCN | MYCN | POLD1 | EIF1AX | SOX2 | MSH3 | MYD88 | RYBP |
| HRAS | FLT3 | NCOR1 | RAD50 | EPHA3 | SOX9 | MYCL | PIK3C3 | SETD2 |
| IDH1 | FLT4 | NFE2L2 | RAD51 | EPHA5 | SUFU | MYOD1 | PIK3CD | SHQ1 |
| IDH2 | GATA3 | NOTCH3 | RAD51B | EPHA7 | SUZ12 | NKX2-1 | PIK3CG | SMARCB1 |
| KIT | GNAS | NOTCH4 | RAD51D | EPHB1 | SYK | NSD1 | PRKCI | SOX17 |
| KRAS | HGF | PIK3CB | RAD52 | FAM46C | TBX3 | P2RY8 | RHOA | SPOP |
| MAP2K1 | IGF1R | PIK3R2 | RAD54L | FANCF | TET1 | PAK1 | SF3B1 | TET2 |
| MAPK1 | JAK1 | PIK3R3 | RECQL4 | FANCG | TMEM127 | PAK7 | SMO | TOPI |
| MET | JAK2 | PPP2R1A | SDHB | FAS | TNFAIP3 | PARK2 | SRC | XRCC2 |
| MTOR | JAK3 | PTPN11 | SDHC | FOXL2 | TNFRSF14 | PARP2 | STAT3 | RAD21 |
| NF1 | KDR | RAF1 | SDHD | FOXP1 | TP63 | INPP4A | TMPRSS2 | STAG2 |
| NF2 | KEAP1 | RASA1 | WT1 | GATA1 | TRAF7 | IRF4 | U2AF1 | IL8 |
| NOTCH1 | MAP2K2 | RIT1 | IL10 | GATA2 | TSHR | KLF4 | ETV6 | INHBA |
| NRAS | MDM2 | RNF43 | IL7R | GNA13 | WHSC1 | LMO1 | RICTOR | PGR |
| NTRK1 | MEN1 | RPTOR | RAD51C | GRIN2A | WHSC1L1 | LYN | ROS1 | PIK3R1 |
| NTRK2 | MLH1 | SMAD2 | SMARCA4 | H3F3A | XIAP | MAP3K13 | RB1 | PDCD1 |
| NTRK3 | MSH2 | SMAD3 | TSC1 | HIST1H1C | XPO1 | MCL1 | RET | TERT |
| PDGFRA | MSH6 | SPEN | VEGFA | HIST1H3B | ERCC1 | POLE | IKBKE | TP53 |
| PIGF | MYC | TGFBR1 | PDGFRB | ICOSLG | pANCD2 | PTCH1 | IKZP1 | PALB2 |
| PIK3CA | NOTCH2 | SMAD4 | PMS2 | ID3 | NT5C2 | PTEN | RAC1 | PBRM1 |
| NUP93 | | | | | | | | |

Those skilled in the art can clearly understand that, for convenience and concise description, only the division of the above-mentioned program modules is used as an example for illustration. In practical applications, the above-mentioned function assignment may be realized by different program modules as required, that is, an internal structure of a device is divided into different program units or modules to complete all or part of the above-mentioned functions. The program modules in the examples may be integrated into one processing unit, or each of the units may exist alone physically, or two or more units may be integrated into one unit. The above integrated unit may be implemented either in the form of hardware or in the foim of software program units. In addition, the specific names of program modules are provided only for the convenience of distinguishing each other, and are not intended to limit the protection scope of the present invention.

FIG. 5 is a schematic structural diagram of the terminal device provided in an example of the present invention. As shown in the figure, the terminal device 200 includes: a processor 220, a memory 210, and computer programs 211 that are stored in the memory 210 and can be running on the processor 220, such as: associated programs for the method for detecting TMB based on capture sequencing. When the processor 220 executes the computer programs 211, the steps in the above examples of the method for detecting TMB based on capture sequencing are implemented, or when the processor 220 executes the computer programs 211, the function of each module in the above examples of the device for detecting TMB based on capture sequencing is implemented.

The terminal device 200 may be a notebook, a palmtop computer, a tablet computer, a mobile phone, and so on. The terminal device 200 may include, but is not limited to, a processor 220 and a memory 210. Those skilled in the art can understand that FIG. 5 shows only an example of the terminal device 200, does not constitute a limitation to the terminal device 200, and may include more or less components than that shown in the figure, a combination of some components, or different components. For example, the terminal device 200 may also include input and output devices, display devices, network access devices (NADs), buses, etc.

The processor 220 may be a central processing unit (CPU), and may also be another general-purpose processor, a digital signal processor (DSP), an application specific integrated circuit (ASIC), a field-programmable gate array (FPGA) or another programmable logic device, a discrete gate, a transistor logic device, a discrete hardware component, etc. The general-purpose processor 220 may be a microprocessor or any conventional processor.

The memory 210 may be an internal storage unit of the terminal device 200, such as a hard disk or a memory of the terminal device 200. The memory 210 may also be an external storage unit of the terminal device 200, such as: a plug-in hard disk, a smart media card (SMC), a secure digital (SD) card, and a flash card that are equipped on the terminal device 200. Further, the memory 210 may also include both an internal storage unit and an external storage unit of the terminal device 200. The memory 210 is configured to store the computer programs 211 and other programs and data required by the terminal device 200. The memory 210 may also be configured to temporarily store data that has been output or will be output.

In the above examples, the description of the examples each has a focus, and portions not described or recorded in detail in one example may refer to related description in other examples.

Those of ordinary skill in the art may be aware that units and algorithm steps in examples described with reference to the examples disclosed herein can be implemented by electronic hardware or a combination of computer software and electronic hardware. Whether these functions are implemented by using hardware or software depends on the specific application of the technical solutions and design constraints. Those skilled in the art may use different methods to implement the described functions for each specific application, but such implementation should not be considered to be beyond the scope of the present invention.

It should be understood that the device/terminal device and method disclosed in the examples of the present invention may be implemented in other manners. For example, the described examples of the devices/terminal devices are merely provided schematically. For example, the division of modules or units merely refers to logical function division, and there may be other division manners in actual implementation. For example, a plurality of units or components may be combined or integrated into another system, or some features may be ignored or not executed. In other respects, the intercoupling or direct coupling or communication connection shown or discussed may be indirect coupling or communication connection through some interfaces, devices, or units; or may be implemented in electrical, mechanical, or other forms.

The units described as separate parts may or may not be physically separate. Parts shown as units may or may not be physical units, which may be located in one position, or may be distributed on a plurality of network units. Some or all of the units may be selected according to actual needs to achieve the objectives of the solutions of the examples.

In addition, functional units in the examples of the present invention may be integrated into one processing unit, or each of the units may exist alone physically, or two or more units may be integrated into one unit. The above integrated unit may be implemented either in the form of hardware or in the form of software functional units.

The integrated module/unit, if implemented in the form of a software functional unit and sold or used as a stand-alone product, may be stored in a computer-readable storage medium. Based on such understanding, all or some of processes for implementing the method in the foregoing examples according to the present invention can be completed by sending instructions to relevant hardware through computer programs 211. The computer programs 211 may be stored in a computer-readable storage medium. When the computer programs 211 are executed by the processor 220, steps of the method in the above examples may be implemented. The computer programs 211 may include computer program codes, and the computer program codes may be in the form of source code, the form of object code, an executable file, some intermediate forms, or the like. The computer-readable storage medium may include: any entity or device capable of carrying computer program 211 codes, a recording medium, a USB disk, a mobile hard disk, a magnetic disk, an optical disc, a computer memory, a read-only memory (ROM), a random access memory (RAM), an electrical carrier signal, a telecommunications signal, a software distribution medium, and the like. It should be noted that, the content included in the computer-readable storage medium may be added or deleted properly according to requirements of the legislation and the patent practice in the jurisdiction. For example, in some jurisdictions, depending on the legislation and the patent practice, the computer-readable storage medium may not include the electrical carrier signal or the telecommunications signal.

It should be noted that the above examples can be freely combined as required. The above descriptions are merely preferred implementations of the present invention. It should be noted that a person of ordinary skill in the art can make several improvements and modifications without departing from the principle of the present invention, and such improvements and modifications should be deemed as falling within the protection scope of the present invention.

What is claimed is:

1. A device for detecting tumor mutation burden (TMB) based on a capture sequencing, comprising:
a panel design module, wherein the panel design module is configured to uniformly add population single-nucleotide polymorphism (SNP) sites to a genome and screen out genome regions, and the genome regions show a highest consistency with whole exome sequencing (WES);
a data acquisition module, wherein the data acquisition module is configured to acquire tissue and plasma samples of a target object and acquire sequencing data of the tissue and plasma samples based on the genome regions screened out by the panel design module;
an alignment module, wherein the alignment module is configured to align the sequencing data acquired by the data acquisition module with a reference genome to acquire mutation data results;
a somatic mutation analysis module, wherein the somatic mutation analysis module is configured to perform a somatic analysis on the mutation data results obtained by the alignment module to obtain somatic mutation results;
a filtering module, wherein the filtering module is configured to remove unreal mutation sites from the somatic mutation results obtained by the somatic mutation analysis module to obtain real somatic mutation sites; and
a calculation module, wherein the calculation module is configured to calculate the TMB according to a number of the real somatic mutation sites obtained by the filtering module.

2. The device according to claim 1, wherein
the panel design module comprises a uniform site design unit and a region screening unit, wherein, the uniform site design unit is configured to screen out the genome regions for designing probes according to a first preset rule and then uniformly add the population SNP sites, and the population SNP sites are screened out according to a second preset rule, and the region screening unit is configured to screen out the genome regions, and the genome regions show the highest consistency with the WES by machine learning on exons;
the first preset rule comprises: removing gaps and regions with a mappability lower than 40 in the genome; and/or after the genome is divided according to a preset window and a step size, removing regions with a GC content higher than 30% and lower than 60%; and/or removing regions with a corresponding preset length, wherein the corresponding preset length comprises a preset number of sites with an Asian population heterozygosity greater than a preset threshold; and
the second preset rule comprises: SNP sites with the Asian population heterozygosity greater than the preset threshold; SNP sites meeting Hardy-Weinberg equilibrium; and/or extending each of the SNP sites to a preset size on both sides to obtain a region and aligning the region with the reference genome, counting a number of positions in the region, wherein the positions are aligned to the reference genome, and removing regions with a number greater than the preset threshold.

3. The device according to claim 1, wherein
the data acquisition module comprises an acquisition unit and a quality control unit, wherein, the acquisition unit is configured to acquire raw data of the tissue and plasma samples of the target object, and the quality control unit is configured to perform a quality control on the raw data of the tissue and plasma samples separately to obtain the sequencing data; and/or
the alignment module comprises a first alignment unit and a second alignment unit, wherein, the first alignment unit is configured to align the sequencing data with the reference genome to obtain an alignment result file, and the second alignment unit is configured to subject the alignment result file to a de-redundancy and a re-alignment in terms of InDel regions to obtain the mutation data results.

4. The device according to claim 1, wherein the device further comprises a specific baseline building module, and the specific baseline building module is configured to build different sequencing depth baselines and tumor fraction baselines for different sequencing depth intervals, sample types, and tumor fraction intervals.

5. The device according to claim 4, wherein
the somatic mutation analysis module is configured to perform the somatic analysis on the mutation data results obtained by the alignment module with VarDict or MuTect2 to obtain the somatic mutation results; or
the somatic mutation analysis module is configured to select a corresponding sequencing depth baseline according to a sequencing depth and a sample type of the tissue and plasma samples and acquire the somatic mutation results based on an in silico germline subtraction algorithm.

6. The device according to claim 4, wherein
the filtering module is configured to filter according to annotation results of the somatic mutation results obtained by the somatic mutation analysis module to remove the unreal mutation sites and obtain the real somatic mutation sites; and
a filtering rule of the filtering module comprises: removing in silico germline mutations according to sample types; filtering out sites with an annotated frequency less than 5% and an occurrence frequency more than 0.2% in a population database; filtering out known tumor-driven gene mutations; filtering out mutation sites manifested as non-germline sites with a predetermined population frequency; filtering out repeat regions or false positive sites generated from an alignment of homologous regions according to a pre-built noise baseline of FFPE sample feature sequence-specific error (SSE); filtering out panel of normal (PoN) sites with a frequency less than a sum of a mean value and 5-fold standard deviation for PoN sites; filtering out preset black-listed sites, wherein the preset black-listed sites have an occurrence frequency greater than 30% in populations or the preset black-listed sites have a population frequency greater than 20% in two sample types of FFPE samples, plasma samples, and blood cell samples; and/or screening out mutations meeting depth requirements according to a sequencing depth baseline of the different sequencing depth baselines and screening out mutations meeting a tumor fraction according to a tumor fraction baseline of the different tumor fraction baselines.

7. A method for detecting TMB based on a capture sequencing, comprising the following steps:
uniformly adding population SNP sites to a genome and screening out genome regions, wherein the genome regions show a highest consistency with WES;
acquiring tissue and plasma samples of a target object and acquiring sequencing data of the tissue and plasma samples based on the genome regions screened out;
aligning the sequencing data with a reference genome to acquire mutation data results;
performing a somatic analysis on the mutation data results to obtain somatic mutation results;
removing unreal mutation sites from the somatic mutation results to obtain real somatic mutation sites; and
calculating the TMB according to a number of the real somatic mutation sites.

8. The method according to claim 7, wherein
the step of uniformly adding population SNP sites to the genome and screening out the genome regions comprises: after the genome regions for designing probes are screened out according to a first preset rule, uniformly adding the population SNP sites screened out according to a second preset rule;
the first preset rule comprises: removing gaps and regions with a mappability lower than 40 in the genome; and/or after the genome is divided according to a preset window and a step size, removing regions with a GC content higher than 30% and lower than 60%; and/or removing regions with a corresponding preset length, wherein the corresponding preset length comprises a preset number of sites with an Asian population heterozygosity greater than a preset threshold; and
the second preset rule comprises: SNP sites with the Asian population heterozygosity greater than the preset threshold; SNP sites meeting Hardy-Weinberg equilibrium; and/or extending each of the SNP sites to a preset size on both sides to obtain a region and aligning the region with the reference genome, counting a number of positions in the region, wherein the positions are aligned to the reference genome, and removing regions with a number greater than the preset threshold.

9. The method according to claim 7, wherein
the step of uniformly adding population SNP sites to the genome and screening out the genome regions further comprises:
counting a number of mutations in exons of the genome of each sample, selecting the exons according to a TMB value on the WES of the each sample to obtain selected exons, and ranking the selected exons based on importance;
starting from a most important exon, adding a marked exon in sequence according to the ranking, and calculating a TMB value of an exon set after each addition and a correlation of the TMB value with a corresponding TMB value obtained from the WES to obtain a calculated correlation; and
according to the calculated correlation, screening out the genome regions with the highest consistency with the WES.

10. The method according to claim 7, wherein
the step of acquiring the tissue and plasma samples of the target object and acquiring the sequencing data of the tissue and plasma samples based on the genome regions screened out comprises:
acquiring raw data of the tissue and plasma samples of the target object, and
performing a quality control on the raw data of the tissue and plasma samples separately to obtain sequencing data; and/or
the step of aligning the sequencing data with the reference genome to acquire the mutation data results comprises:
aligning the sequencing data with the reference genome to obtain an alignment result file, and
subjecting the alignment result file to a de-redundancy and a re-alignment in terms of InDel regions to obtain the mutation data results.

11. The method according to claim 7, wherein the method for detecting the TMB further comprises a step of building different sequencing depth baselines and tumor fraction baselines for different sequencing depth intervals, sample types, and tumor fraction intervals.

12. The method according to claim 11, wherein
the step of performing the somatic analysis on the mutation data results to obtain the somatic mutation results comprises: performing the somatic analysis on the mutation data results obtained by the alignment module with VarDict or MuTect2 to obtain the somatic mutation results; or
the step of performing the somatic analysis on the mutation data results to obtain the somatic mutation results comprises:
selecting a corresponding sequencing depth baseline according to a sequencing depth and a sample type of the tissue and plasma samples; and
acquiring the somatic mutation results based on an in silico germline subtraction algorithm.

13. The method according to claim 11, wherein
the step of removing the unreal mutation sites from the somatic mutation results to obtain the real somatic mutation sites comprises: filtering according to annotation results of the somatic mutation results obtained by the somatic mutation analysis module to remove the unreal mutation sites and obtain the real somatic mutation sites; and
a filtering rule of the filtering module comprises: removing in silico germline mutations according to sample types; filtering out sites with an annotated frequency less than 5% and an occurrence frequency more than 0.2% in a population database; filtering out known tumor-driven gene mutations; filtering out mutation sites manifested as non-germline sites with a predetermined population frequency; filtering out repeat regions or false positive sites generated from alignment of homologous regions according to a pre-built noise baseline of FFPE sample feature sequence-specific error (SSE); filtering out panel of normal (PoN) sites with a frequency less than a sum of a mean value and 5-fold standard deviation for PoN sites; filtering out preset black-listed sites, wherein the preset black-listed sites have an occurrence frequency greater than 30% in populations or the preset black-listed sites have a population frequency greater than 20% in two sample types of FFPE samples, plasma samples, and blood cell samples; and/or screening out mutations meeting depth requirements according to a sequencing depth baseline of the different sequencing depth baselines and screening out mutations that meet a tumor fraction according to a tumor fraction baseline of the different tumor fraction baselines.

14. A terminal device, comprising a memory, a processor, and computer programs, wherein the computer programs are stored in the memory and are running on the processor, wherein, when the computer programs are running on the processor, the steps of the method for detecting the TMB based on the capture sequencing according to claim 7 are implemented.

15. A computer-readable storage medium, wherein the computer-readable storage medium stores computer programs, wherein, when the computer programs are executed by a processor, the steps of the method for detecting the TMB based on the capture sequencing according to claim 7 are implemented.

16. The device according to claim 2, wherein the device further comprises a specific baseline building module, and the specific baseline building module is configured to build different sequencing depth baselines and tumor fraction baselines for different sequencing depth intervals, sample types, and tumor fraction intervals.

17. The device according to claim 3, wherein the device further comprises a specific baseline building module, and the specific baseline building module is configured to build different sequencing depth baselines and tumor fraction baselines for different sequencing depth intervals, sample types, and tumor fraction intervals.

18. The method according to claim 8, wherein
the step of uniformly adding population SNP sites to the genome and screening out the genome regions further comprises:
counting a number of mutations in exons of the genome of each sample, selecting the exons according to a TMB value on the WES of the each sample to obtain selected exons, and ranking the selected exons based on importance;
starting from a most important exon, adding a marked exon in sequence according to the ranking, and calculating a TMB value of an exon set after each addition and a correlation of the TMB value with a corresponding TMB value obtained from the WES to obtain a calculated correlation; and
according to the calculated correlation, screening out the genome regions with the highest consistency with the WES.

19. The method according to claim 8, wherein the method for detecting the TMB further comprises a step of building different sequencing depth baselines and tumor fraction baselines for different sequencing depth intervals, sample types, and tumor fraction intervals.

20. The method according to claim 10, wherein the method for detecting the TMB further comprises a step of building different sequencing depth baselines and tumor fraction baselines for different sequencing depth intervals, sample types, and tumor fraction intervals.
